(19) United States
(12) Patent Application Publication
(10) Pub. No.: US 2007/0173860 A1
(43) Pub. Date: Jul. 26, 2007

(54) Plunger

(76) Inventor: Takuya Iwasaki, Tokyo (JP)

Correspondence Address: ALCON, IP LEGAL, TB4-8, 6201 SOUTH FREEWAY, FORT WORTH, TX, 76134, US

(21) Appl. No.: 11/336,043

(22) Filed: Jan. 20, 2006

(51) Int. Cl. A61F 9/00

(52) U.S. Cl. 606/107; 623/6.12

(57) ABSTRACT

A plunger having blunt, rounded tip offset from the centerline of the plunger rod and having a notch. The offset tip assures that the tip is biased downward against the bottom of the cartridge bore. Such a downward bias helps prevent the tip from riding up over the IOL and being folded within the IOL. The notch provides a space for the haptic to rest while the IOL is being pushed down the bore of the cartridge so that the haptic does not get trapped between the bore sidewall and the plunger tip.

### BACKGROUND OF THE INVENTION

This invention relates to intraocular lenses (IOLs) and more particularly to plungers used to inject an IOL into an eye.

The human eye in its simplest terms functions to provide vision by transmitting and refracting light through a clear outer portion called the cornea, and further focusing the image by way of the lens onto the retina at the back of the eye. The quality of the focused image depends on many factors including the size, shape and length of the eye, and the shape and transparency of the cornea and lens. When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial lens or IOL. More recently, IOLs are being used in purely refractive procedures where patients may not have a discernable cataract, but where there has been a loss of accommodation.

While early IOLs were made from hard plastic, such as polymethylmethacrylate (PMMA), soft, foldable IOLs made from silicone, soft acrylics and hydrogels have become increasingly popular because of the ability to fold or roll these soft lenses and insert them through a smaller incision. Several methods of rolling or folding the lenses are used. One popular method is an injector cartridge that folds the lenses and provides a relatively small diameter lumen through which the lens may be pushed into the eye, usually by a soil tip plunger. The most commonly used injector cartridge design is illustrated in U.S. Pat. No. 4,681,102 (Bartell), and includes a split, longitudinally hinged cartridge. Similar designs are illustrated in U.S. Pat. Nos. 5,494,484 and 5,499,987(Feingold) and 5,616,148 and 5,620,450(Eagles, et al.). In an attempt to avoid the claims of U.S. Pat. No. 4,681,102, several solid cartridge, have been investigated, see for example U.S. Pat. No. 5,275,604(Rheinish, et al.) and 5,653,715(Reich, et al.).

These prior art devices used plungers that were substantially symmetrical about a longitudinal axis and generally contained a cylindrical or flared soft tip that completely filled the bore of the injector cartridge. See, for example, U.S. Pat. Nos. 4,681,102(Bartell) and 4,919,130 (Stoy, et al.), and WIPO Publication No. WO 96/29956, the entire contents of which are incorporated herein by reference. Other plungers had hooked or forked tips meant to grasp the edge of the IOL. See for example, U.S. Pat. Nos. 4,573,998(Mazzocco), 5,494,484 and 5,499,484(Feingold), 5,616,148 and 5,620,450(Eagles, et al.) and 5,653,715(Reich, et al.), the entire contents of which are incorporated herein by reference. One plunger tip recently commercially introduced is designed so that the IOL rolls around the tip as the IOL is advanced down the cartridge. See U.S. Pat. No. 5,735,858(Makker, et al.), the entire contents of which is incorporated herein by reference.

While these symmetric plunger tip designs work well with robust, rubbery, elastic lens materials, the performance of these tip designs is less than optimal when used with an IOL produced with the characteristics of a viscoelastic material, such as a soft acrylic. In particular, the tip design that encourages the haptic on multipiece and single piece IOLs to get caught between the tip and the inner lumen of the bore in the cartridge, thereby damaging the haptic and the optic, in some instances. Accordingly, a need continues to exist for an IOL injector plunger designed to optimize performance when used with an IOL made from a viscoelastic material, e.g., a hydrophobic acrylic.

### BRIEF SUMMARY OF THE INVENTION

The present invention improves upon prior art lens injector plungers by providing a plunger having a blunt, rounded tip offset from the centerline of the plunger rod and having a notch. The offset tip assures that the tip is biased downward against the bottom of the cartridge bore. Such a downward bias helps prevent the tip from riding up over the IOL and being folded within the IOL. The notch provides a space for the haptic to rest while the IOL is being pushed down the bore of the cartridge so that the haptic does not get trapped between the bore sidewall and the plunger tip. An additional improvement upon the prior art is that the IOL optic and haptics are all managed at the most forward tip of the plunger/cartridge, allowing the IOL to be inserted into its final position in the capsular bag in one step rather than multiple steps that increases the chances of infection.

It is accordingly an object of the present invention to provide a lens injector plunger having a rounded tip containing a notch.

It is a further object of the present invention to provide a lens injector plunger having an offset tip.

It is a further object of the present invention to provide a lens injector plunger that minimizes the potential for damage to the optic and/or the haptics.

Other objectives, features and advantages of the present invention will become apparent with reference to the drawings, and the following description of the drawings and claims.

### BRIEF DESCRIPTION OF THE DRAWINGS

FIG. 1 is a partial side elevational view of the lens injector plunger of the present invention. FIG. 2 is a top plan view of the lens injector plunger of the present invention. FIG. 3 is a front elevational view of the lens injector plunger of the present invention. FIG. 4 is a partial cross-sectional view of the intraocular lens injector of the present invention.

### DETAILED DESCRIPTION OF THE INVENTION

As best seen in FIG. 4, intraocular lens injectors suitable for use with the present invention generally consist of handpiece 12, cartridge 10 and plunger 14. Cartridge 10 contains bore 16 having a bore wall 28 through which the intraocular lens (not shown) is pushed by plunger 14 until the lens exist bore 16 at nozzle 18. Cartridge 10 and handpiece 12 may be of any suitable design well-known in the art.

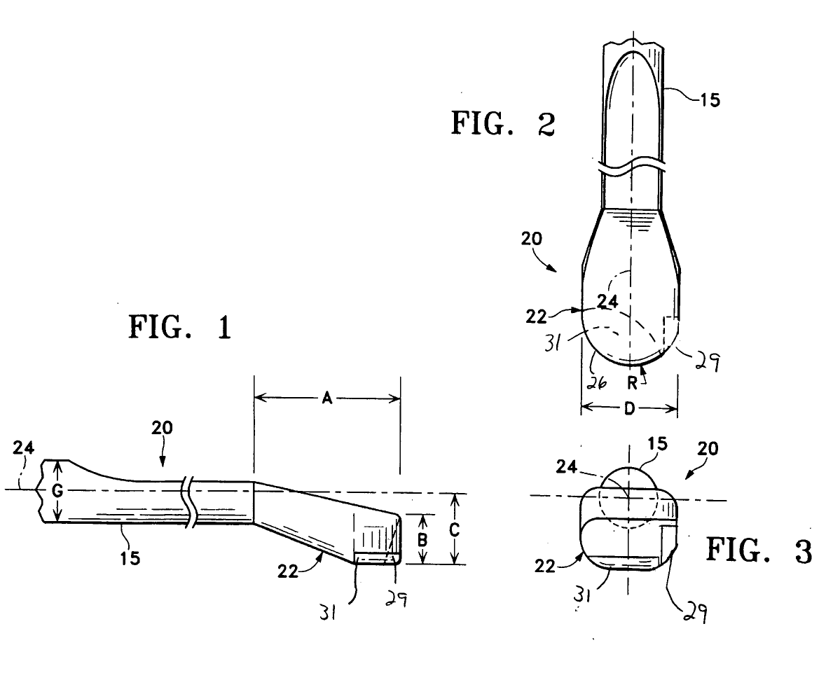

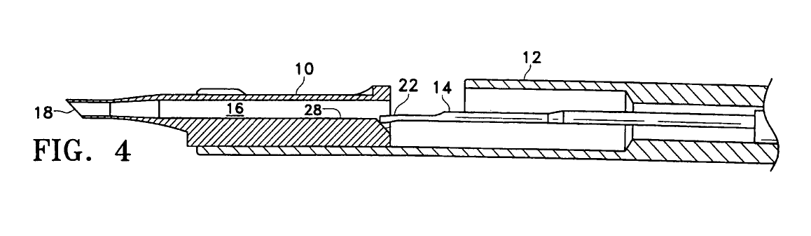

As best seen in FIGS. 1-3 plunger 14 contains tip 20 that contains IOL contacting portion 22 that is off set asymmetrically relative to longitudinal centerline 24. Portion 22 also contains convexly rounded nose 26. Length A of portion 22 preferably is between 0.10 inches and 0.20 inches, with 0.127 inches being preferred. Width D of portion 22 preferably is between 0.060 inches and 0.100 inches, with 0.083 inches being most preferred. The radius R of nose 26 may be any suitable dimension, with between 0.030 inches and 0.050 inches being preferred and 0.042 inches being most preferred. Tip 20 also contains notch 29 that extends through the entire thickness B of tip 20, but extends only partially across width D of tip 20. Notch 29 preferably is cut through thickness B of tip 20 at an angle. Notch 29 provides an opening into which the IOL haptic (not shown) can fall. Tip 20 also contains arcuate recess 31 that allows tip 20 to receive and engage the edge of the IOL optic (not shown) when the IOL haptic is within notch 29. As opposed to notch 29, recess 31 extends substantially along width D of tip 20 but only partially into thickness B of tip 20.

The offset distance C from centerline 24 will vary depending upon the diameter of bore 16 and plunger rod 15 and the thickness B of portion 22. By way of example, when bore 16 has a diameter of around 0.100 inches, plunger rod 15 has a diameter G of around 0.060 inches and thickness B of portion 22 is between 0.030 inches and 0.060, offset C generally will be between 0.047 inches and 0.077 inches.

While certain embodiments of the present invention have been described above, these descriptions are given for purposes of illustration and explanation. Variations, changes, modifications and departures from the systems and methods disclosed above may be adopted without departure from the scope or spirit of the present invention.

I claim:

1. An intraocular lens injection system, comprising:
a) a handpiece having a plunger, the plunger having a longitudinal centerline;
b) an injection cartridge having a bore, the cartridge to be received in the handpiece so that the plunger can travel down the bore;
c) a tip on the plunger, the tip having a notch extending for the full thickness of the tip but extending only partially across the width of the tip.

2. The intraocular lens injection system of claim 1 wherein the notch is cut through the thickness of the tip at an angle.

3. The intraocular lens injection system of claim 1 wherein the tip further comprises a recess that extends substantially across the width of the tip, but only partially through the thickness of the tip.

4. An intraocular lens injection system, comprising:
a) a handpiece having a plunger, the plunger having a longitudinal centerline;
b) an injection cartridge having a bore, the cartridge to be received in the handpiece so that the plunger can travel down the bore;
c) a tip on the plunger, the tip having a notch and a recess, the notch extending for the full thickness of the tip but extending only partially across the width of the tip and the recess extending substantially across the width of the tip, but only partially through the thickness of the tip.
